(19) United States
(12) Patent Application Publication
(10) Pub. No.: US 2021/0065922 A1
(43) Pub. Date: Mar. 4, 2021

(54) Emission monitoring system for a venting system of a nuclear power plant

(71) Applicant: FRAMATOME GmbH, ERLANGEN (DE)

(72) Inventor: Axel Hill, Stockstadt (DE)

(21) Appl. No.: 16/969,867

(22) PCT Filed: Feb. 20, 2019
(86) PCT No.: PCT/EP2019/054251

§ 371 Date: Aug. 13, 2020

(30) Foreign Application Priority Data
Feb. 22, 2018 (DE) 10 2018 202 702.1

(51) Int. Cl. G21C 17/00; G21C 13/02; G21C 13/10; G21C 9/004; G01N 1/22; G01N 33/00; G01N 27/62

(52) U.S. Cl. CPC G21C 17/00; G21C 13/022; G21C 13/10; G21C 9/004; G01N 2001/2223; G01N 1/2247; G01N 33/0055; G01N 27/62; G01N 1/2202

(57) ABSTRACT

A nuclear system, in particular a nuclear power plant (2), includes a containment (4) and an associated venting system (6), which has a venting line (8) connected to the containment (4), and an emission monitoring system (16) is provided for the venting system (6). A representative measuring sample is taken from the clean gas line of the venting system, and can be tested for aerosol-type decomposition products online in a subsequent analysis system. The emission monitoring system comprises a sampling line (44) for a sample flow branching off from the venting line (8) and leading into a sample container (32), and a recirculation line (54) leading from the sample container (32) to the venting line (8). The sample container (32) contains a wet scrubber (34) for the sample flow, as well as an ionisation separator (64) downstream of the wet scrubber (34) in relation to the sample flow. A liquid removal line (78) leads from the sample container (32) to an analysis unit (20).

The present disclosure relates to a nuclear facility, in particular, a nuclear power plant, having a containment, an associated venting system and having an emission monitoring system for monitoring the emissions of the venting system. The present disclosure furthermore relates to a method for the emission monitoring of a venting system in a nuclear facility.

### BACKGROUND

In the event of a severe accident of a nuclear power plant, in addition to the release of steam and hydrogen the release of large amounts of radioactive fission products into the containment can occur. Due to the high energy input into the containment and the release of non-condensing gases, an over pressure failure of the containment shell, which constitutes the last barrier for the retention of fission products into the environment, is no longer excluded. This is the case, in particular, in the case of relatively small inertized boiling water reactor containments (typical volumes 5,000 to 20,000 m³). The release of the non-condensable hydrogen together with steam leads to a rapid rise in pressure, which goes beyond the design pressure and can go up to the failure pressure of the containment.

In order to prevent an over pressure failure of the containment, older and newer nuclear power plants are equipped with a filtered pressure relief of the containment after the accidents in Chernobyl and Fukushima. In spite of the filtering, a release of gaseous and aerosol-bound radioactive fission products into the environment occurs to a certain extent during the pressure relief, depending on the effectiveness of the filter devices.

The International Atomic Energy Agency (IAEA) and local safety authorities require an emission monitoring of the activity released during the pressure relief. Here, in addition to other fission products, due to the long-term soil contamination, in particular, cesium and the iodine that is dose-relevant for the population (in organically bound and elemental form) come into the focus of the authorities. Also, the personnel, who are present in the facilities at the time of the accident, could be impacted, in particular, by the noble gases, which cannot be retained, and require an improved protection.

The data on the released activity are in principle required for the information of the population and the authorities for the derivation of accident measures and, for example, an evacuation and setting up of a safety zone. In this connection, above all, a rapid online provision of the essentially important measurement data is required.

A subsequent more precise and more detailed chemical and radiological analysis can be made with a so-called evidence preservation filter in the laboratory. The fission products retained in the filter can be differentiated into iodine (organic and elemental) and into aerosol-bound radionuclides. In a precise analytical division of the sample in the laboratory a determination of the released components is possible. The laboratory analysis, however, requires a substantially greater amount of time and is not suitable for timely decisions.

The release is therefore usually measured and recorded by an emission monitoring system connected or coupled to the venting system. Such an emission monitoring system is known, for example, from US 2016/0118149 A1.

In addition to the analytical method for determining the released gaseous and aerosol-bound fission products, the provision of an as representative a sample as possible of the medium to be measured in the analyzer is of crucial importance. It should thereby be noted that an aerosol sampling of the clean gas flow of the venting system must have a higher degree of separation than the filter devices of the pressure relief system. With the sampling systems from the prior art this objective cannot be achieved, in any event not in a satisfactory way.

### SUMMARY

A problem addressed by the present disclosure is to indicate a device and an associated method for obtaining a representative measurement sample, which is taken from the clean gas line of the venting system, and which can be measured in a subsequent analysis system online for aerosol fission products.

A nuclear facility is provided, in particular a nuclear power plant, having a containment and having an associated venting system including a venting line connected to the containment. An emission-monitoring system is present, having a sampling line for a probe flow, branching from the venting line and leading into a sample container, and a return line leading from the sample container to the venting line. The sample container comprises a wet scrubber for the probe flow and an ionization separator which is switched downstream of the wet scrubber in relation to the probe flow. A liquid-tapping line leads from the sample container to an analyzing unit.

Advantageous embodiments are the subject matter of the following detailed description.

The following description discloses a special sampling method and an associated sampling system, in short, a sampler, with which it is made possible to take a representative sample from the venting gas flow and to analyze the latter in an online measurement method for aerosol-bound fission products and gaseous fission products. The invention, in particular, is aimed at the range of very fine aerosols, which are to be expected after the filter- or cleaning stages of the venting system. However, at the same time, the sampler is also suitable for the deposition of large aerosols, so that a malfunction of the venting system (for example, failure of a filter stage) can also be detected online.

The sampler comprises a scrubber region or wet scrubber for the deposition of aerosols in the separating grain size range of preferably 0.1 to 1.0 μm. The scrubbing liquid can, for this purpose, be conditioned with chemical substances, for example, sodium thiosulfate, in order to bind iodine. Furthermore, the wettability of the solid aerosol particles and their deposition are thereby improved. For the deposition of aerosols in the scrubber region one or a plurality of venturi tubes preferably immersed in the scrubbing liquid can be used. The liquid-to-gas ratio in the venturi tube is preferably 0.5 to 10.

The gas precleaned in the scrubber region then flows into an ionization deposition region or, in short, ionization separator. The latter consists substantially of a high voltage field with a preferably centrally arranged spraying electrode and a depositing electrode. The container wall of the sampling vessel or, in short, sample vessel, can serve as the depositing electrode. The electrical ionization field is formed between the emitting negative spraying electrode with a high voltage, for example, of 10 to 80 kV and the grounded depositing electrode. The solid- and liquid particles of the aerosol dispersion are electrostatically charged by ions and electrons, which are generated in the corona of the spray wire under high DC voltage. The particles and aerosols still located in the gas of the probe flow are negatively charged and migrate in the electrical field to the deposit surface (positive pole). By means of the electrical ionization field the degrees of separation <0.1 to 0.01 μm can be reached, which lie far above those of the filter of the containment venting system. The high degree of separation makes it possible to be able to monitor the separation effectiveness of the venting system. By means of periodic spraying with the aid of a liquid spraying system, the deposit surfaces are cleaned and the aerosols are transferred to the scrubbing liquid.

The cleaned gas is fed back into the venting line, specifically, preferably downstream of a throttle switched into the venting line, the so-called venting throttle. The pressure difference generated via the venting throttle makes possible the sampling flow (passive drive). In the sampling line guiding the probe flow, more precisely stated, in the sample return line, a flow throttle is also located parallel to the venting line, also referred to as the sample throttle. With the throttle in the sample line it is ensured that the same pressure prevails in the sampling vessel as in the venting line. The vaporization and evaporation of the scrubbing liquid is hereby prevented. By means of a supercritical approach flow of the throttle in the sample line, the volume flow is kept constant via the separator, so that the scrubber unit with the venturi tube and the ionization separator can operate in the optimal deposition range.

The scrubbing liquid from the sampler is continuously or periodically fed to an analyzing unit for nuclide-specific online measurement. The analyzing unit can in this connection comprise a spectrometer, for example, a germanium spectrometer or fluorescence spectrometer. Only by the transfer of the aerosols to the scrubbing liquid is it possible to guide the medium to be measured over a large distances in a sampling line to the analyzer, without inadmissible deposits of the aerosols in the sampling line occurring. The analyzing unit can in this connection be placed at a sufficient distance to the radiating sampling location.

Optionally, a gaseous sample is taken upstream of the sample throttle and is guided to the analyzer. The analyzed sample is fed back again downstream of the sample throttle. The gaseous sample is conveyed passively, namely by the pressure difference to the sample throttle.

The activity collected in the scrubbing liquid can be used at the end of the venting process as an evidence preservation filter of the integrally released activity.

For the further optimization of the aerosol deposition two or more samplers can be switched in series.

In summary, the concept according to the present invention comprises a sampling vessel with an integrated upstream wet separation, preferably with a venturi scrubber, and an ionizing electrical fine separation stage of the aerosol dispersion. The return of the fine aerosols into the scrubbing liquid makes possible a loss-minimizing transport of the aerosols to the analysis device. An additional gas sampling takes place preferably passively by using the pressure difference generated via a throttle. The probe flow is advantageously kept constant by the supercritical operation of the throttle, so that the operating point of the sampler is designed in an optimized manner and can be kept constant. Thus, the relevant balancing of the released fission products in relation to the venting flow is possible.

The advantages of this concept are summarized in particular as follows:
A representative aerosol- and gas sampling is made possible after the filtered pressure relief in the venting system.
Consequently, a precise online monitoring of the released aerosol-bound and gaseous fission products can take place.
Thus, a monitoring of the separation effectiveness of the filtered pressure relief can occur.
Only small aerosol deposits form in the sample lines.
The analyzers can be placed in a protected manner at a distance from the sample location.
A universal use of the analyzers available on the market is possible.
The concept is independent of the method of the filtered pressure relief (for example, dry filtering, wet separation).

### BRIEF SUMMARY OF THE DRAWINGS

Several embodiments of the invention are elucidated below by means of the schematic and greatly simplified drawings.

FIG. 1 in part shows in the manner of an overview a nuclear power plant with a venting system for filtered pressure relief in the event of a severe accident and with an associated emission monitoring system.

FIG. 2 shows a first variant of the emission monitoring system from FIG. 1 in an enlarged representation.

FIG. 3 shows a second variant of the emission monitoring system.

### DETAILED DESCRIPTION

The same or identically acting elements are provided with the same reference signs in all of the figures.

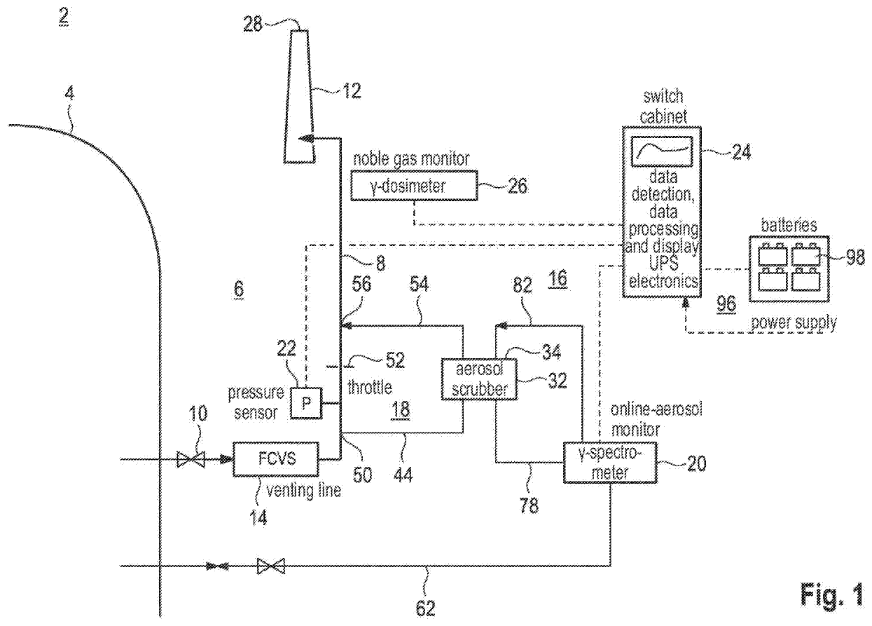

The nuclear facility partially shown in FIG. 1 is a nuclear power plant 2, for example, of the pressurized water reactor or boiling water reactor type, with a safety shell referred to as containment 4, which encloses the nuclear components and in the normal case shields it hermetically from the environment. To control severe accidents with release of radioactive fission products and with significant pressure build-up in the containment 4, a filtered pressure relief is provided with the aid of an associated pressure relief system or venting system 6 (FCVS=Filtered Containment Venting System). For this purpose, a pressure relief line or venting line 8 is connected to the containment 4, by means of which when necessary—after the opening of an associated shut-off valve 10—the pressurized containment atmosphere can be conducted into the open, for example, via a chimney 12. A number of filter units 14 or filter stages for the pressure relief flow or venting flow is switched into the venting line 8, in order to minimize the radioactive contamination of the environment during the venting process. These can be dry filters, wet filters, wet scrubbers, molecular sieves and the like or any combination thereof.

In order to ensure that the filter units 14 function properly during the venting process and in order to metrologically detect a possible radioactive residual contamination of the environment, an emission monitoring system 16 is installed in the facility according to FIG. 1. The emission monitoring system 16 comprises a sampling system 18, which extracts a probe from the filtered venting flow downstream of the filter units 14—therefore from the so-called clean gas flow—, and supplies the same to an analyzing unit 20 for determining the radiological activity contained therein. The analyzing unit 20 can comprise, in particular, a spectrometer or another device for determining the nuclide-specific activity. With the aid of further measured variables, for example, the pressure P in the venting line 8 detected by a pressure sensor 22, and/or by suitable estimates of the mass flow or volume flow in the venting line 8, the entire (nuclide-specific) activity released into the environment can be determined in an associated electronic evaluation unit 24 and can be visualized on a display device, preferably in real time (online monitoring).

Of course, even more measured values can be included in the evaluation, for example, from a dosimeter 26, which, for example, is positioned in the vicinity of the venting line 8 or its outlet 28. The power supply 96 for the evaluation unit 24 and the analyzing unit 20 and any other electrical devices that may be present (for example, a high voltage generator, see further below) is preferably designed so as to be autonomous and fail-safe, for example, by means of batteries 98 or accumulators.

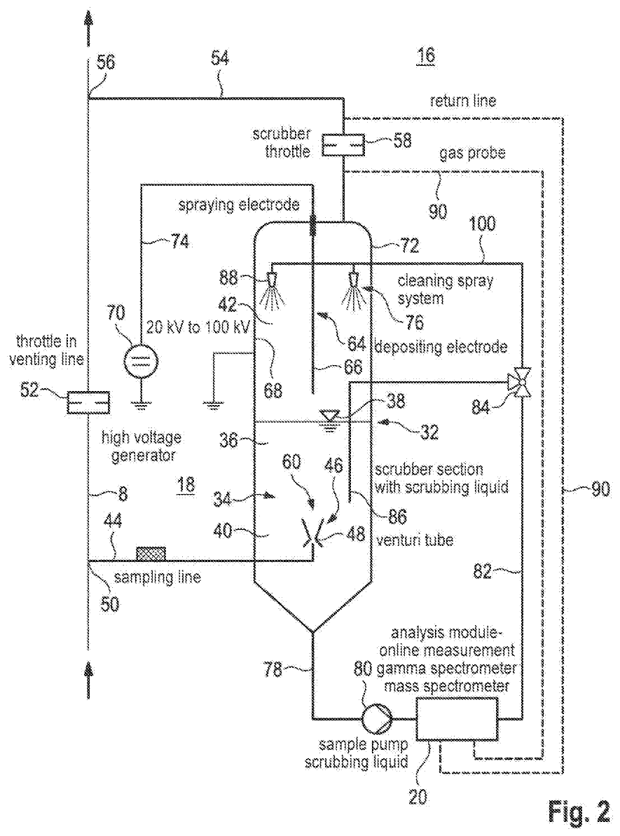

In the emission monitoring that portion of the activity is of interest in particular, which stems from the aerosols entrained in the venting flow, especially the particularly small particles or suspended particles, which are not retained or only insufficiently in the filter unit 14. The emission monitoring system 16 described here is therefore optimized to a particular degree for obtaining a representative aerosol probe from the clean gas flow, which is elucidated in detail below by means of FIG. 2.

A component of the emission monitoring system 16 depicted in FIG. 2 is a sampling system 18, which has a sample container 32 with an integrated wet scrubber 34. The sample container 32 is a container closed on all sides in a pressure-tight and media-tight manner, here in the example of cylindrical shape and arranged so as to stand upright. In a lower region of the sample container 32 a scrubbing liquid chamber 36 is located, which is filled during the operation up to a predetermined filling level 38 with a scrubbing liquid 40. A gas chamber 42 is located above it.

Downstream of the filter unit 14 (see FIG. 1) a sampling line 44 branches from the venting line 8 and leads into the sample container 32. More precisely, the sampling line 44 opens into the scrubbing liquid chamber 36 at its end, wherein the outlet is advantageously designed in the manner of a venturi tube 46. Several venturi tubes 46 can be present in the parallel circuit in terms of flow. The respective venturi tube 46 preferably has a narrow point or fillet point 48 with an opening in the nozzle wall, via which the inner lying flow channel communicates with the surrounding scrubbing liquid 40. If the probe flow flows through the venturi tube 46, a suction or entrainment of the surrounding scrubbing liquid 40 therefore occurs through the opening at the fillet point 48.

Downstream of the branching 50 of the sampling line 44, a throttle 52 is switched into the venting line 8, which is also referred to as a venting throttle. In addition, a return line 54 leads from the gas chamber 42 of the sample container 32 back into the venting line 8, wherein the junction 56 lies downstream of the throttle 52. A throttle 58, which is as referred to as a sample throttle, is also switched into the return line 54.

By means of the described arrangement a part of the venting flow branches from the venting line 8 and is led as probe flow through the sampling line 44 into the sample container 32. The branched partial flow passes through the venturi tube 46, wherein it is mixed or interacts intimately with the scrubbing liquid 40 sucked in or entrained in the region of the fillet point 48. This mixture is discharged into the scrubbing liquid 40 at the nozzle outlet 60 lying below the liquid level. Alternatively, the blowing out can occur directly into the gas chamber 42. Due the intimate interaction of the venting flow with the scrubbing liquid 40 the entrained aerosols are incorporated into the scrubbing liquid 40. The degree of separation is particularly high when venturi tubes are used. However, alternatively, other outlet nozzles or outlet openings are also conceivable.

Furthermore, condensable gas components of the venting flow are deposited in the sample container 32 partially in liquid form, whereby the filling level 38 in the venting operation tends to rise. In order to prevent an excessive rise, a return of scrubbing liquid 40 from the sample container 32 into the containment 4 can be provided via a liquid return feed line 62 indicated only in FIG. 1. Alternatively, excessive scrubbing liquid 40 can be discharged into a separate collection container.

After the separation of liquid falling downwards and gas rising upwards brought about by gravity, the cleaned gaseous partial flow of the venting flow is collected in the gas chamber 42 of the sample container 32 and flows upwards through the return line 54, in order finally to be united again with the main flow at the junction 56 of the return line 54 into the venting line 8. The junction 56 is located downstream of the throttle 52 with respect to the venting line 8. The pressure conditions are set by the throttle 52 in the venting line 8 and the throttle 58 in the return line 54 such that the branching and later reunification of the parallel partial flows does not require further driving means such as pumps or the like, but rather is driven exclusively by the overpressure in the containment 4 relative to the ambient atmosphere.

In order to increase the separation efficiency, in particular, in respect to fine aerosols with comparatively small particle size, an electrical ionization separator 64 (also referred to as an electrostatic separator, corona separator or electrostatic filter) is connected downstream of the wet scrubber 34 integrated into the sample container 32, which electrical ionization separator is advantageously also integrated into the sample container 32, specifically into the gas chamber 42 above the scrubbing liquid chamber 36. The wet scrubber 34 is accordingly to be regarded as a coarse separator or first separator stage, and the ionization separator 64 forms a fine separator or a second separator stage.

The ionization separator 64 comprises at least one spraying electrode 66 and one depositing electrode 68, between which a voltage difference in the high voltage range, for example, of 20 kV to 100 kV is applied during the operation by means of a high voltage generator. The high voltage generator 70 is expediently arranged outside the sample container 32 and is connected to the spraying electrode 66 via an electrically insulated connection cable 74 guided through the container wall 72. The spraying electrode 66 and the depositing electrode 68 are located in the gas chamber 42 of the sample container 32. The depositing electrode 68 can also be formed, as here in FIG. 2, by the (grounded) metallic container wall 72 of the sample container 32. By means of the electrons and ions migrating from the spraying electrode 66 to the depositing electrode 68, the aerosol particles entrained in the gas flow are ionized and migrate in the electrical discharge field (corona) to the depositing electrode 68, on the surface of which they are deposited or settle. With the aid of a cleaning system or spraying system 76 that sprays liquid, the deposited aerosols are continuously or at time intervals, in particular, periodically, backwashed from the depositing electrode 68 into the scrubbing liquid 40 of the wet scrubber 34. Alternatively, or additionally a rapping mechanism can be provided for the cleaning.

The venturi tubes 46 are advantageously immersed deep enough in the scrubbing liquid 40, so that there is no disturbing discharge upwards into the region of the ionization separator 64. The preferred mode of operation of the spraying system 76 is a continuous spraying operation.

A liquid tapping line 78 is connected to the sump of the sample container 32, preferably at its deepest point, into which liquid tapping line a feed pump 80 is connected, and which leads further downstream to the analyzing unit 20, which, comprises, for example, a gamma spectrometer and/or a mass spectrometer. When the feed pump 80 is switched on, a sample of the scrubbing liquid 40 located in the sample container 32 with the aerosols contained therein is thus conveyed to the analyzing unit 20 and is measured there with regard to its radiological activity.

The liquid sample is conveyed back into the sample container 32 through a liquid return line 82 guided by the analyzing unit 20 into the sample container 32. Advantageously, the liquid tapping line 78 merges into the liquid return line 82 on/in/at the analyzing unit 20. Both lines together can therefore be regarded as a continuous recirculation line, so that a single feed pump 80 is sufficient for the sample transport. The liquid sample is therefore to a certain extent guided past the analyzing unit 20 and is analyzed there preferably “on the fly”. Deviating from the example depicted here, the feed pump 80 can also be arranged downstream of the analyzing unit 20.

In the example according to FIG. 2, the liquid return line 82 branches into two partial strands at a line branching 84. One of said partial strands leads directly back to the scrubbing liquid 40, therefore has an outlet 86 arranged in the scrubbing liquid chamber 36. The other forms a feed line 100 for the at least one spray nozzle 88 of the spraying system 76 arranged preferably as high as possible in the gas chamber 42 above the depositing electrode 68. The line branching 84 is advantageously designed as a controllable, switchable 3-way valve, in order to be able to set the flow rate through the line strands as required.

In an advantageous variant, moreover, a gas probe line 90 is present, which is connected on the input side downstream of the ionization separator 64, but still upstream of the throttle 58 to the gas chamber 42 of the sample container 32 or to the return line 54. In the further course, the gas probe line 90 is guided past the analyzing unit 20 and is connected on the output side downstream of the throttle 58 to the return line 54 or upstream of the throttle 52 directly to the venting line 8. For the adaptation or optimization of the pressure conditions a throttle can also be arranged in the gas probe line 90. In this way, a gas probe of the gas flow can be taken and analyzed in a passive way, after said gas flow has passed through the two separator stages (wet scrubber 34 and ionization separator 64) within the sample container 32 and the aerosols contained therein have been separated into the scrubbing liquid 40.

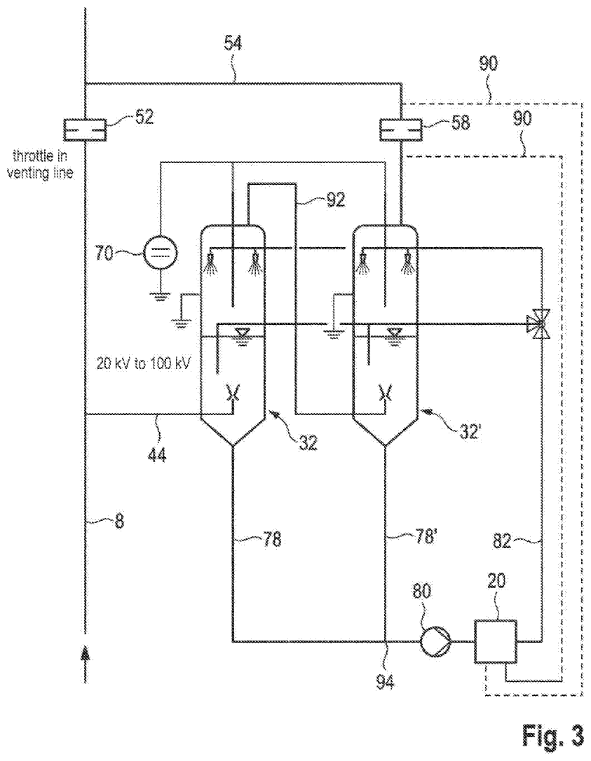

In the variant depicted in FIG. 3, two sample containers 32, 32′ of the type known from FIG. 2 are switched in series with respect to the probe flow. That means, the probe flow branched from the venting flow in the venting line 8 initially enters through the sampling line 44 into the first (here on the left) sample container 32 and there, as described in connection with FIG. 2, passes through the two aerosol separator stages (wet scrubber and ionization separator). The gas flow that has already in this manner been largely freed of aerosols is subsequently fed via the connection line 92 to the second (here on the right) sample container 32′ and there passes in an analogous manner through two aerosol separator stages, therefore is once again depleted with regard to the aerosol content. Finally, the probe flow leaves the sample container 32′ via the return line 54 with the throttle 58 and is united again with the venting flow at the junction 56 of the return line 54 into the venting line 8.

As in the manner described in connection with FIG. 2, a liquid tapping line 78, 78′ is connected to each of the two sample containers 32, 32′, wherein these two lines are united at the union 94. That means, the liquid samples from both sample containers 32, 32′ are combined. The sample mixture is subsequently—driven by the feed pump 80—guided past the analyzing unit 20 and finally distributed (see also FIG. 2) via a system of liquid return lines 82 to the respective spraying system 76 and to the outlet 86 opening in each case directly into the scrubbing liquid 40 in both sample containers 32, 32′. In this respect, some variations are possible: In an alternative design, the liquid samples from the two sample containers 32, 32′ can, for example, be guided past the analyzing unit 20 separately from one another and be returned to the respective original sample container 32, 32′.

### LIST OF REFERENCE SIGNS

2 nuclear power plant
4 containment
6 venting system
8 venting line
10 shut-off valve
12 chimney
14 filter unit
16 emission monitoring system
18 sampling system
20 analyzing unit
22 pressure sensor
24 evaluation unit
26 dosimeter
28 outlet
32, 32′ sample container
34 wet scrubber
36 scrubbing liquid chamber
38 filling level
40 scrubbing liquid
42 gas chamber
44 sampling line
46 venturi tube
48 fillet point
50 branch
52 throttle
54 return line
56 junction
58 throttle
60 nozzle outlet
62 liquid return feed line
64 ionization separator
66 spraying electrode
68 depositing electrode
70 high voltage generator
72 container wall
74 connection cable
76 spraying system
78, 78′ liquid tapping line
80 feed pump
82 liquid return line
84 line branching
86 outlet
88 spray nozzle
90 gas probe line
92 connection line
94 union
96 power supply
98 battery
100 feed line

What is claimed is:

1-15. (canceled)

16. A nuclear facility comprising:
a containment;
a venting system associated with the containment including a venting line connected to the containment;
an emission-monitoring system comprising:
a sampling line for a probe flow, the sampling line branching from the venting line and leading into a sample container,
a return line leading from the sample container to the venting line, the sample container including a wet scrubber for the probe flow and an ionization separator downstream of the wet scrubber in relation to the probe flow, a liquid-tapping line leading from the sample container to an analyzing unit.

17. The nuclear facility of claim 16, wherein the wet scrubber is in a lower part of the sample container and the ionization separator is thereabove in an upper part of the sample container.

18. The nuclear facility of claim 16, wherein the wet scrubber is a venturi scrubber.

19. The nuclear facility of claim 18, wherein the venturi scrubber includes a venturi tube completely immersed in a scrubbing liquid.

20. The nuclear facility of claim 16, wherein the ionization separator includes a spraying electrode arranged in a gas chamber and a depositing electrode formed by a wall of the sample container.

21. The nuclear facility of claim 16, further comprising a number of filter units is switched into the venting line, and wherein the branch of the sampling line from the venting line is located downstream of the filter units.

22. The nuclear facility of claim 16, further comprising a throttle provided into the venting line, between the branch of the sampling line from the venting line and the junction of the return line into the venting line.

23. The nuclear facility of claim 16, further comprising a throttle is arranged in the return line.

24. The nuclear facility of claim 23, wherein the throttle in the return line is configured for a supercritical approach flow.

25. The nuclear facility of claim 23, further comprising a gas-probe line branching from the return line upstream of the throttle, the gas-probe line leading to the analyzing unit.

26. The nuclear facility of claim 25, wherein the gas-probe line runs into the return line again downstream of the throttle.

27. The nuclear facility of claim 16, wherein the liquid-tapping line merges into a downstream of the analyzing unit, the liquid-return line leading into the sample container.

28. The nuclear facility of claim 27, wherein the liquid-return line or a branch line branching off therefrom forms a feed line for a spraying system arranged in the sample container for backwashing aerosols accumulated on the ionization separator into the wet scrubber.

29. The nuclear facility of claim 16, further comprising a further sample container provided with a further wet scrubber and a further ionization separator, the sample container and the further sample container being in series in relation to the probe flow.

30. The nuclear facility of claim 16, wherein the nuclear facility is a nuclear power plant.

31. A method for emission monitoring of a venting system of a nuclear facility comprising:
extracting an aerosol probe from the venting stream by:
providing a probe flow branching off from the venting stream,
cleaning the probe flow in a wet scrubber so aerosols contained in the probe flow are separated into a scrubbing liquid,
passing the probe flow then through an ionization separator so aerosols contained in the probe flow are separated and fed into the scrubbing liquid, and
feeding a probe of the scrubbing liquid containing aerosols bound therein to an analyzing unit.

32. The method according to claim 31, wherein the nuclear facility is a nuclear power plant.
